(19) United States
(12) Patent Application Publication
(10) Pub. No.: US 2022/0076849 A1
(43) Pub. Date: Mar. 10, 2022

(54) SYSTEM AND METHOD FOR ANALYZING USER HEALTH

(71) Applicant: R Zero, Inc., Ridgeland, MS (US)

(72) Inventors: Frederick Kennedy Sones, Madison, MS (US); Timothy Allan Clark, Flowood, MS (US); Matthew Johnson Kenedy, Ridgeland, MS (US); Huey Jiun Ngo, Brandon, MS (US)

(73) Assignee: R Zero, Inc., Ridgeland, MS (US)

(21) Appl. No.: 17/478,560

(22) Filed: Sep. 17, 2021

Related U.S. Application Data
(63) Continuation-in-part of application No. 16/172,351, filed on Oct. 26, 2018.
(60) Provisional application No. 62/577,541, filed on Oct. 26, 2017.

(51) Int. Cl. G16H 50/80; G16H 50/20; G16H 10/20

(52) U.S. Cl. CPC G16H 50/80; G16H 10/20; G16H 50/20

(57) ABSTRACT

A system and method for analyzing health data includes providing a user one or more surveys, compiling user input from the one or more surveys, storing user notes, collecting information regarding external factors, analyzing the user input relative to the information regarding the environmental factors, and generating output for the user based on the analysis of the user input relative to the information regarding the environmental factors where the output may be location specific.

### CROSS-REFERENCE TO RELATED APPLICATION

This application is a continuation in part of U.S. patent application Ser. No. 16/172,351 filed Oct. 26, 2018, which claims priority to U.S. Provisional Application No. 62/577,541 filed Oct. 26, 2017, both of which are incorporated herein by reference in their entirety.

### FIELD OF THE INVENTION

The present invention is generally directed toward a system and method for analyzing disease transmission patterns and analyzing user health data.

### BACKGROUND OF THE INVENTION

It is widely known that many health issues can be attributed to environmental factors. Asthma, for example, is triggered by, among other environmental factors, high amounts of pollen or other fine particulate matter. In another study, Geoffrey Martin of the University of Cincinnati published an article in 2013 suggesting lightning strikes could trigger migraines. Environmental exposure to blue-green algae has even been attributed as a cause of ALS.

It would be beneficial to have a tool that could help track disease development and symptoms in relationship to environmental factors and alert affected users. For example, alerting asthma patients that there are high levels of pollen could help them avoid extended time outdoors, which may reduce their asthma attacks. None of the current tools for monitoring environmental factors and alerting a user to these factors take into account all of the most impactful environmental factors, the changes of such factors, and feedback from a user regarding how the user feels as it relates to the user's health condition.

Other diseases are caused by highly contagious pathogens. For example, smallpox is incredibly infectious and a huge percentage of Americans are susceptible. By identifying and quarantining people exposed to infectious smallpox particles before they become infectious, officials could prevent an epidemic spread. However, the standard epidemiological method for studying the disease progression through a society is to interview the infected people. This method relies on trying to recreate a timeline from memory, which may be unreliable.

It would be beneficial to have a tool that could recreate an individual's exact location history to provide accurate information to doctors and public health officials. For example, Tuberculosis can be latent in a person for months or years. If a doctor could look at a patient's location history and determine where the infection began, he could prevent future cases from developing.

### SUMMARY OF THE INVENTION

We disclose herein a system and method to help scientists identify how diseases develop and environmental factors that may result in symptoms. This software toolkit contains tools for analyzing spatiotemporal factors pertaining to health in real-time and retrospect for individuals and populations.

These and other features, aspects, and advantages of the present disclosure will become better understood with reference to the following description and appended claims. The accompanying drawings, which are incorporated in and constitute a part of this specification, illustrate embodiments of the invention and, together with the description, serve to explain the principles of the invention.

### BRIEF DESCRIPTION OF THE DRAWINGS

A full and enabling disclosure of the present disclosure, including the best mode thereof, directed to one of ordinary skill in the art, is set forth in the specification, which makes reference to the appended figures.

FIG. 1 depicts a layout of the system architecture.

FIG. 2 depicts a graphical health timeline.

FIG. 3 depicts a schematic of Timeline Alignment.

FIG. 4 depicts a representation of the modules in the system.

FIG. 5 depicts a representation of the pathogen exposure.

FIG. 6 depicts an exemplary screenshot of the toolkit in use

FIG. 7 depicts another exemplary screenshot of the toolkit in use.

FIG. 8 depicts an exemplary quantitative health questionnaire utilized in the system.

FIG. 9 depicts a block diagram of a software system for a mobile application, according to various examples.

FIG. 10A depicts a short survey displayed within the mobile application of FIG. 9, according to various examples.

FIG. 10B depicts a selection screen for choosing a long survey within the mobile application of FIG. 9, according to various examples.

FIG. 11A depicts a first exemplary question for a long survey within the mobile application of FIG. 9, according to various examples.

FIG. 11B depicts a second exemplary question for a long survey within the mobile application of FIG. 9, according to various examples.

FIG. 11C depicts a third exemplary question for a long survey within the mobile application of FIG. 9, according to various examples.

FIG. 11D depicts a fourth exemplary question for a long survey within the mobile application of FIG. 9, according to various examples.

FIG. 12A depicts a first exemplary question for a condition specific survey within the mobile application of FIG. 9, according to various examples.

FIG. 12B depicts a second exemplary question for a condition specific survey within the mobile application of FIG. 9, according to various examples.

FIG. 12C depicts a third exemplary question for a condition specific survey within the mobile application of FIG. 9, according to various examples.

FIG. 12D depicts a fourth exemplary question for a condition specific survey within the mobile application of FIG. 9, according to various examples.

FIG. 12E depicts a fifth exemplary question for a condition specific survey within the mobile application of FIG. 9, according to various examples.

FIG. 13 depicts a display of a scaled index score generated from user input to the mobile application of FIG. 9, according to various examples.

FIG. 14A depicts a graph of past and predicted scaled index scores generated from user input to the mobile application of FIG. 9, according to various examples.

FIG. 14B depicts a list of past user input to the mobile application of FIG. 9, according to various examples.

FIG. 15 is a block diagram of a software system for a mobile application, according to various examples.

FIG. 16 is a schematic diagram of an API of a software system for a mobile application, according to various examples.

FIG. 17 is a schematic diagram of a mobile application of the API of FIG. 16, according to various examples.

Repeat use of reference characters in the present specification and drawings is intended to represent the same or analogous features or elements of the present disclosure.

### DETAILED DESCRIPTION

The following detailed description is presented to enable any person skilled in the art to make and use the invention. For purposes of explanation, specific details are set forth to provide a thorough understanding of the present invention. However, it will be apparent to one skilled in the art that these specific details are not required to practice the invention. Descriptions of specific applications are provided only as representative examples. Various modifications to the preferred embodiments will be readily apparent to one skilled in the art, and the general principles defined herein may be applied to other embodiments and applications without departing from the scope of the invention. The present invention is not intended to be limited to the embodiments shown, but is to be accorded the widest possible scope consistent with the principles and features disclosed herein.

We disclose herein tools to study how space and time affect disease development and symptoms and methods for alerting affected people. This could be used in a variety of ways, such as: to manage diseases triggered by spatiotemporal factors; to prevent the spread of highly contagious pathogens; and to search for the root cause of diseases of unknown origin. Any of these use cases would yield immense societal value. Currently, there is no tool that takes into account all of the most impacting environmental factors, their changes, and a user's providing input or interactions as to how the user feels as it relates to the user's health conditions. The application disclosed herein is configured to use machine learning to take into account all of the most impacting environmental factors, their changes, and user input related to a user's health to predict more accurately over time how a user could potentially feel based on the inputted information.

Our platform combines geospatial data, spatiotemporal data, and health data to identify the potential source of health conditions. The platform combines data through software that analyzes the datasets and then uses statistical analyses to identify significant variables. To get accurate analyses, our system uses individuals' or a population's health records, individuals' location histories, remotely sensed environmental data, hyperlocal environmental data from the disclosed sensors, and other geospatial features.

In one exemplary embodiment, the disclosed platform is a web application that allows for different types of statistical analyses in a modular manner. The basic component of this application is a web interface that loads and visualizes a layer of location histories (including from mobile device location history) and different layers of accessory spatial data (keypoints, temperature rasters, vectors, etc.). It will be understood that the platform is described herein as a web application but may be a web application, a desktop application, any variant of a mobile application, or any comparable application without departing from the scope of the present disclosure.

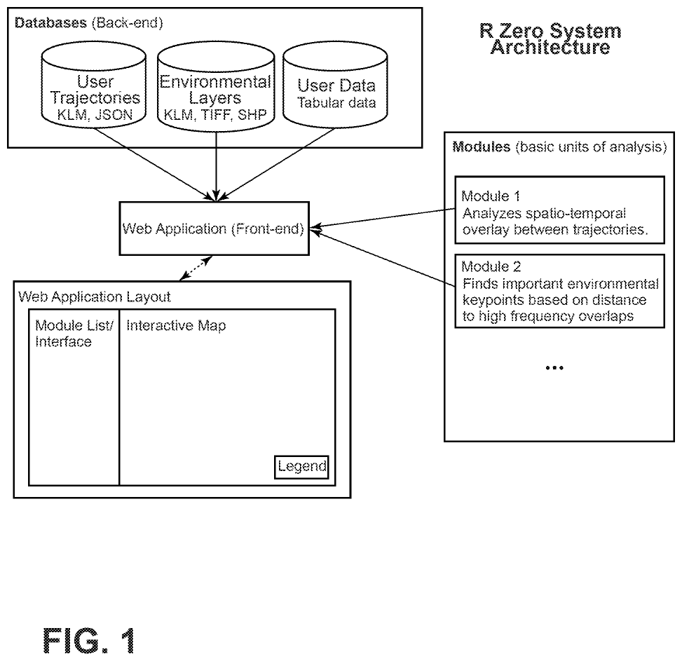

This data is operated on by modules that each performs a specific type of analysis. Each module developed for the application allows for a different type of analysis. In the current embodiment, each module exists as one or two pieces—a JavaScript library and a back-end API (if necessary). However, it will be understood that the pieces are not limited to a JavaScript library and a back-end API and may be any comparable components without departing from the scope of the present disclosure. The separation of module logic from visualization component is preferred, as different types of analyses require vastly different computation resources. For example, a module that trains deep recurrent neural networks to identify periodic behavior in groups of user trajectories will require dedicated computing resources, and should not run on the same system that serves the web interface. Additionally, not all customers will need access to the same set(s) of modules. See FIG. 1 for a general visualization of the system architecture.

Additionally, we disclose products that complement the web application. These include environmental sensors to collect hyperlocal environmental data that can be deployed in a variety of ways, including as weather stations or mobile transmitters; data scrapers to mine information from public sources, such as NASA's satellite portal; and native mobile applications that can be used to collect location history data.

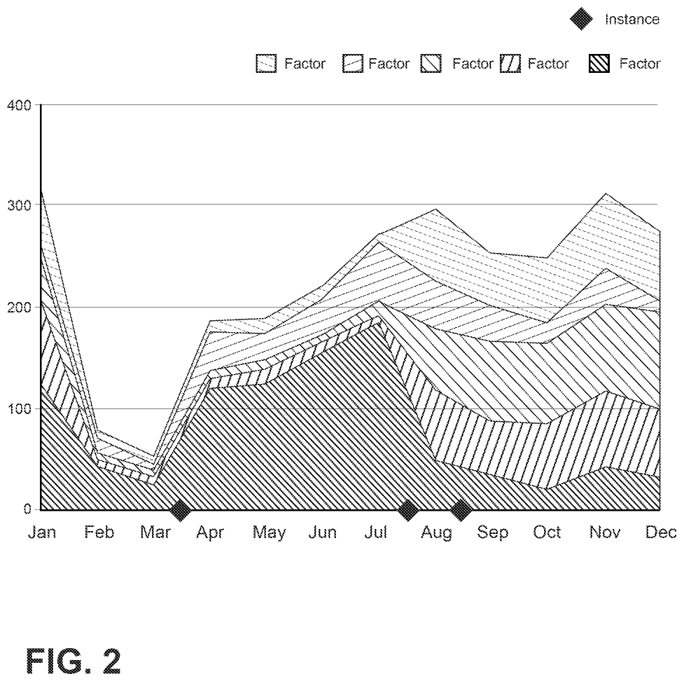

We further disclose a customizable health timeline. As shown in FIG. 2, the health timeline is a graphical way to view a patient's health history in conjunction with environmental factors. In the graph, the diamond shaped marks along the x axis represent the patient's health events and the y axis represents environmental variables. Among other options, the user may change the values and axes of the graph.

It should be appreciated that the system and software toolkit can be developed using any software language, social API, or programmable architecture such as HTML/CSS, PHP, MySQL, Twitter Bootstrap, JS, Python, C/C++, Arduino, Raspberry Pi, Keras, Tensorflow, Scikit-Learn, MongoDB, Docker and Swift.

The Crossings Engine

The Crossings Engine, our name for the web application and technology surrounding it, is the core of the disclosed software platform. The disclosed system combines individual location histories and environmental data from satellites and sensors using this novel Crossings Engine. The Crossings Engine is trained on a library of disease modules to identify potential sources of conditions based on location and environmental data. This allows public health officials to identify sources of outbreaks in near real time.

Web Application Architecture

In an exemplary embodiment, the web application may consist of two parts: a back-end Python server that hosts the static web content and responds to API calls from the web application and the front-end web application that allows a user to interact with data to use modules to analyze it. This design is based around the modular concept described previously, where a single module contains the functionality for performing a basic unit of analysis on data.

In the absence of any modules, the web application provides:

Loading and unloading a list of trajectory files as a single layer

Loading and unloading a list of keypoint files as a single layer

Displaying the keypoints and trajectories graphically, in a GUI similar to other modern mapping software

Highlighting a single trajectory when hovered over with the mouse.

It will be understood that the back-end Python server is exemplary only and that other back-end or front-end application architecture may be used to achieve user interaction with the data without departing from the scope of the present disclosure.

In a potential embodiment of the architecture of the application, the file run_keras_server.py contains the majority of the functionality of the application. The load_model( ) function loads in the saved model that was trained on the initial dataset, compiles it, loads in the weights, and loads in the tokenizer. The prepare_data(data) function receives data, uses the tokenizer to convert the texts in the data to sequences, pads the sequences, and returns the padded sequences. It will be understood that the functionality may be held in other comparable back-end or front-end application architecture (e.g., various modules) without departing from the scope of the present disclosure.

There are three routes set up after the main methods: The first route is for the home page that gives the user an option to navigate to the loadFile page. The second route is the loadFile page. There, the user can upload a j son file containing the patient notes to receive the trajectory prediction. The third route is the prediction route, which receives a flask post request and will return the trajectory predictions. This route may be contacted via API or through our site's User Interface.

Trajectory Slicing and Dicing

A key challenge in analyzing spatiotemporal factors that contribute to health is analyzing where people go in a relevant way. To do this, the disclosed platform inputs user trajectories, reduces them to relevant scopes, and further splits them into useful periods. The trajectories come from a variety of places, such as our app and Google Timeline (https://www.google.com/maps/timeline). These trajectories are typically GeoJSON or KML files, but are sometimes in other formats.

The Crossings Engine considers a few parameters for reduction; based on the disease under examination and date of diagnosis, trajectories typically can be reduced to a matter of weeks or days (rather than years). This is typical, but not guaranteed. For example, consider Legionnaire's Disease and Tuberculosis. Legionnaire's Disease is almost always diagnosed within 20 days of exposure to legionella. Unlike Legionnaire's Disease, Tuberculosis can take years to diagnose, this means the Crossings Engine might need to take in years of location history data to provide valuable output concerning tuberculosis.

The Crossings Engine can then process data into trajectory units in two ways: by day, where the first location-time point of a given day is the beginning point for the trajectory and the last location-time point of the day ends the trajectory, or by “idle threshold” time, where trajectories are created based on contiguous blocks of movement.

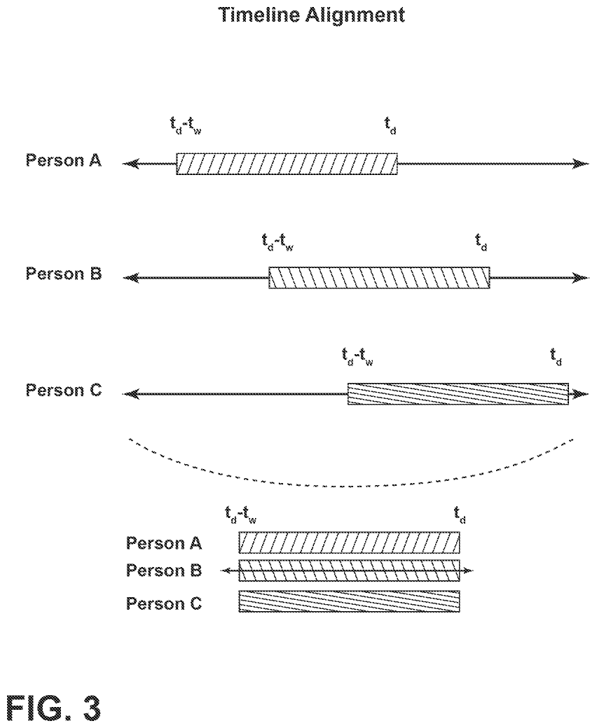

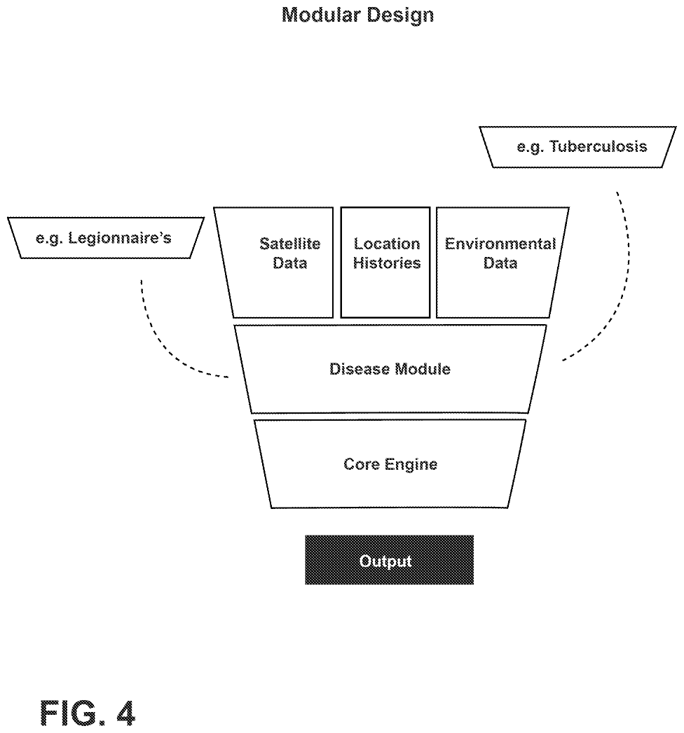

As will be appreciated from FIG. 3, the backend slices and arranges timelines based on the time of diagnosis t_{d} and the disease time window t_{w}. FIG. 4 shows that the Crossings Engine has disease modules that calibrate key variables, such as the time window to examine or the pathogen lifespan. Because of the Crossings Engine's modular design, these parameters can be changed easily and tweaked endlessly.

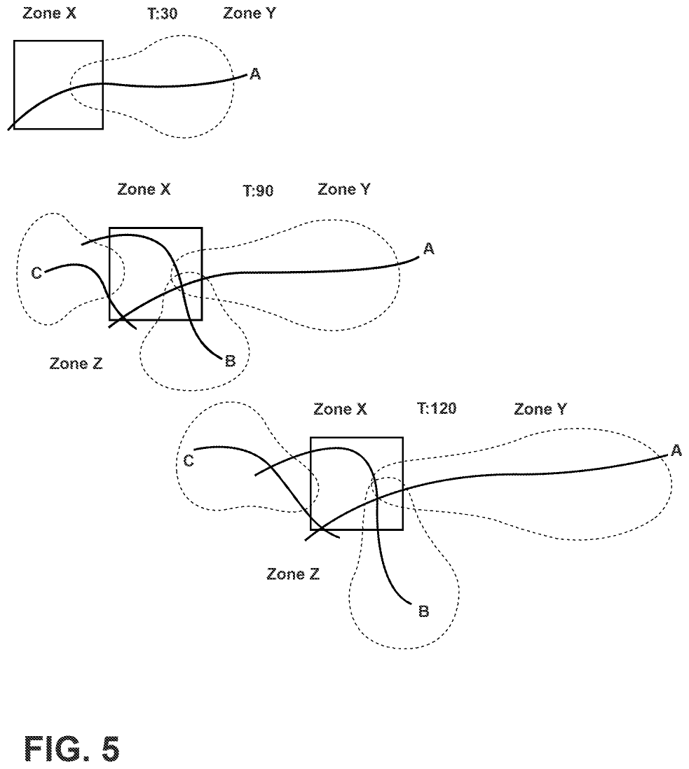

As will be appreciated from FIG. 5, our Crossings Engine Disease Modules allow researchers to investigate specific outbreaks or diseases with specific parameters. For example, the mysterious Pathogen X has the following disease module parameters: Continuous Source Outbreak, Person-to-Person Infectious, Disintegrating Bounding Box. It can then be calculated that:

From t=0 to tₑₓₚₒₛᵤᵣₑ<60, the chance of infection if exposed to the pathogen is 100%.

From t=60 to tₑₓₚₒₛᵤᵣₑ<120, the chance of infection if exposed to the pathogen is 50%.

From tₑₓₚₒₛᵤᵣₑ=120 onward, the chance of infection if exposed to the pathogen is 0%, and

Exposed subjects are immediately infected and infectious.

Once a module and data set are loaded into the Crossing Engine, a machine-learning algorithm identifies the spatiotemporal factors that may be contributing to the development of conditions or diseases.

Keypoint Data

Geographic keypoints are a second important data set for analyzing how spatiotemporal factors contribute to health outcomes. There are many different types of geographic keypoints and many data sources that provide them. For example, restaurants, supermarkets, parks, and highway exits are all common geographic keypoints in geospatial information system analysis. OpenStreetMap is an open-source effort that provides these and many other key points around the world for free. The Crossings Engine allows users to input huge sets of keypoints and use them for analysis, including custom data and data from public sources, such as OpenStreetMap.

Environmental Sensing

Environmental variables provide an important and challenging dimension to spatiotemporal health analysis. Environmental data is collected globally from satellite-borne sensors and locally from weather stations. Much of this data is publicly accessible; NASA and other government entities publish data they collect on web portals regularly. Some other websites, such as Accuweather, allow users to upload data from private weather stations to create large datasets. There are some limits to the effectiveness of this environmental data; for some measurements, rural areas lack local data and are typically represented by projections from the nearest urban center. In addition to collecting data from publicly accessible sources, the disclosed platform provides a way to collect hyperlocal environmental data via environmental transmitters.

Environmental Transmitters

To collect environmental data from rural Mississippi locations, our team created two sets of transmitters. One set of transmitters uses a WiFi connection to send sensor records to a web database through an API. The other set of transmitters uses a GSM (cell phone) data connection to send sensor records to a web database through an API. The data connection is the primary difference between the two embodiments.

The WiFi-based sensor consists of an Arduino MEGA, a Raspberry Pi, an optional GPS unit, and one or more environmental sensors. This transmitter is designed either to be stationary or mounted on a vehicle (in which case a GPS unit would be required). The transmitter uses the Arduino microcontroller to read the GPS and any environmental sensors. The Arduino then sends this data to the Raspberry Pi, which is listening to the Arduino via a USB port and a Python script. The Python Script then processes the data, storing it locally. Another Python script checks for a WiFi connection regularly and, when connected to the internet via WiFi, retrieves all locally stored data, sends it to a web database through a series of API calls, then dumps the local copies of the readings. In theory, this transmitter could store many gigabytes of data. The Raspberry Pi's operating system boots from a microSD card, which also keeps the locally stored data. In the current version of the transmitter, this is a 32 GB microSD card, which would allow for many days of regular environmental readings before encountering memory restrictions.

The GSM-based sensor consists of an Arduino Uno, and Adafruit FONA (GPS and GSM Modules), a GSM SIM card, a GPS Antenna, a GSM antenna, a battery, and one or more environmental sensors. This transmitter is designed to be mounted on a vehicle and powered by a fuse tap connected to the vehicle's fuse panel. As a proof-of-concept, we mounted transmitters on a fleet of buses in two counties in northern Mississippi. The transmitter uses the Arduino microcontroller to read the GPS and the environmental sensor(s), prepare an API-compatible URL, and interact with the FONA module. The FONA processes the URL and reads the data from the API (which typically responds with “OK”—meaning the data was successfully processed by the API). After attempting to send the collected data, the Arduino waits for a programmable number of seconds and then repeats the operation. In the same loop, the Arduino verifies that it has a GPS location lock, a cellular network connection, and a GSM data connection. If any of these fail, the Arduino prioritizes reconnecting before reading the sensor again. This transmitter is mounted inside a 3″ by 5″ case, with the environmental sensors mounted to the outside of the case to allow for sufficient exposure to the environment.

Modules

As described previously, the Crossings Engine has a modular design, where different computational modules add functionality to the basic web application. In this section, the first three modules are described in detail. In general, a module consists of a back-end and a front-end component. The backend code is responsible for registering API endpoints to interact with the front-end code and doing the bulk of the computational analysis. The front-end part of a module is responsible for initializing the computation (via an init( ) method), drawing GUI elements, and handling clicks on the module to execute functionality. The purpose of this separation is to decouple module code from the rest of the functionality of the web application as much as possible. This currently is a key architectural focus of the platform.

Trajectory Similarity Module

The trajectory similarity module compares all of the trajectories loaded into the web application and groups them into clusters of “similar behavior.” Specifically, the module uses the discrete Frechet distance to calculate all trajectory pairs' “similarity.” The discrete Frechet distance between two trajectories is 0 if the trajectories are identical, and grows as the trajectories become more dissimilar. The module uses the computed distances to perform a clustering of the trajectories with agglomerative tree based clustering. This clustering algorithm requires a set parameter of how many clusters to find. This module performs the analysis multiple times for different numbers of clusters in the range [2,10] in order to find the “best” number of clusters. The “goodness” of a particular cluster is evaluated using the silhouette value, which is larger for better clusterings. This module returns the clustering that maximizes the silhouette score.

Trajectory-Keypoint Finder Module

The trajectory-keypoint finder module analyzes which keypoints are “most visited” by the trajectories in the study area. To do this, each keypoint is buffered by some distance then intersected with each trajectory. The number of trajectories that the keypoint intersects with is considered to be the number of times it is “visited” by the trajectory set. The module returns the number of times each keypoint is visited and colors the keypoints in the GUI accordingly.

This module is both useful for some public health epidemiology contexts and an example of how to integrate keypoint and trajectories interworking into the module based analysis framework and could be extended.

Firetower Module

The “Firetower” module analyzes trajectory commonality from the trajectory set. To do this, each trajectory is buffered by some distance to create a polygon and then compared to all other trajectories. The module then creates sets where polygons overlap n number of times. This is a difficult problem because there are a factorial number of intersection operations to perform. Per trajectory, brute force calculation would create an exponentially more difficult (and, more important, computationally expensive) operation. To control for this complication, this module calculates overlaps at (n Choose x) and propagates those. This module returns polygons of overlapping trajectory buffers and then colors them on the GUI based on the number of overlaps.

This module is both useful for some public health epidemiology contexts and an example of how to create trajectory-trajectory comparison within the module based analysis framework.

Usage Examples:

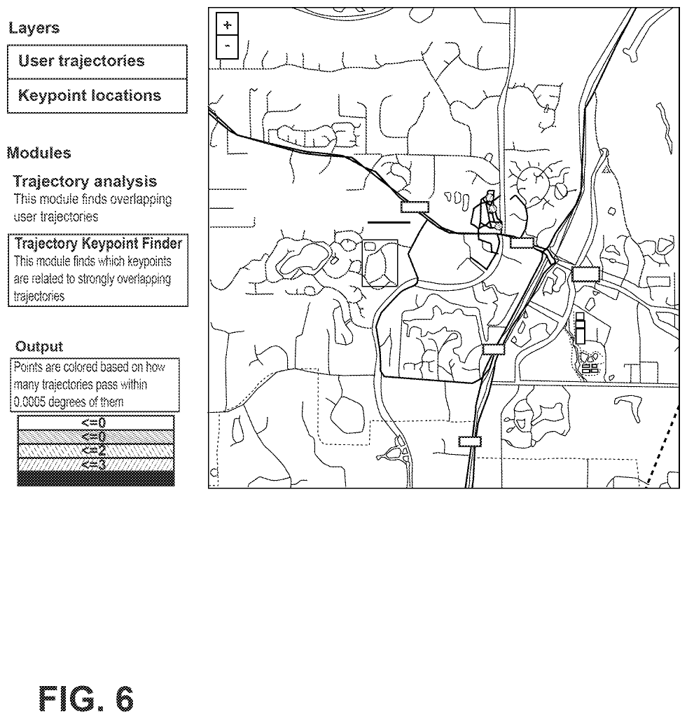

The Crossings Engine takes a set of location histories, environmental data, key points, and a disease module to identify potential sources of infection. FIG. 6 shows an example of the information that can be gleaned from this tool. In this image, the Crossings Engine processed a series of location histories tagged with Legionnaires' Disease and identified key points within Madison, Miss. where the infection could have originated.

We have deployed a suite of environmental sensors across Mississippi. Over the course of this pilot, our sensors have collected over 25,000 environmental readings across more than 200,000 GPS points. The transmitters have been mounted on private vehicles, public school buses, and even drones. After identifying geographic areas with high rates of some condition, these transmitters will allow our team to create highly detailed, hyperlocal pictures of the environment and investigate what environmental factors (if any) are contributing to the condition.

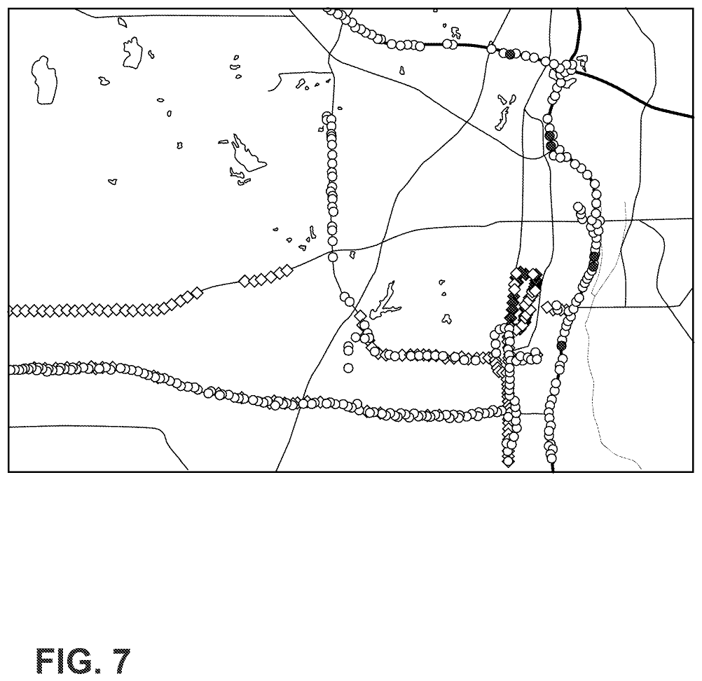

The sensors create hyperlocal maps for many environmental variables. As will be appreciated from FIG. 7, the screenshot shows the VOC levels in Tupelo, Miss. along major roads. The yellow areas are VOC levels that could be dangerous over an extended period of time.

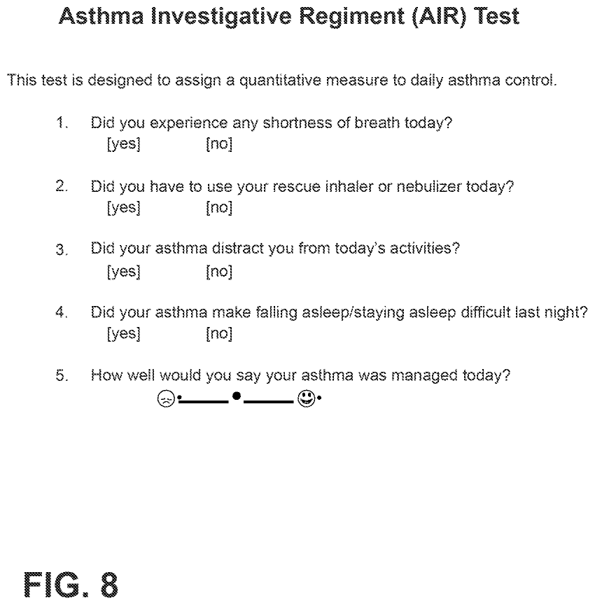

As shown in FIG. 8, data is obtained directly from patients via questionnaires and tracked over time to provide quantitative measures of daily health and specific health-related issues, such as asthma.

In another exemplary embodiment, the disclosed platform may be a mobile application configured to be stored on a user's portable device. In various examples, the mobile application may be configured to include various components of the web application. In other examples, the mobile application may be configured to operate in conjunction with the web application described above. The mobile application may further be configured to combine data about a user and external factors to provide statistical analyses of the user's health. In various examples, the mobile application may be configured to utilize a statistical model to develop a scaled index score. The scaled index score may be related to the probability of a symptom being triggered for the user or to other statistical model predictions related to the user's health and/or symptoms, as discussed in more detail elsewhere herein. It will be understood that the mobile application described herein is exemplary and that the platform may instead be a web application, a desktop application, any variant of a mobile application, or any comparable application without departing from the scope of the present disclosure.

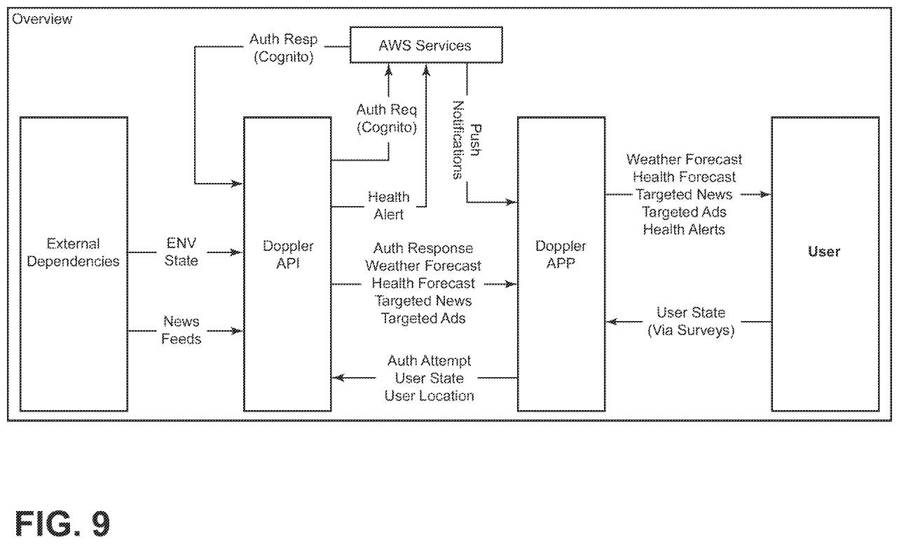

As shown in FIG. 9, the mobile application may be configured to operate in connection with a plurality of software components, including, but not limited to an API and/or cloud-based service components, to further collect and log time information, geographical location information, and/or information regarding environmental factors to detect trends in the user's health relative to the collected information. For example, the mobile application may be configured to track any number of environmental factors (e.g., the mobile application may be configured to log and/or display data about 40 or more environmental factors). These environmental factors may include, for example, wind gust, visibility, solar radiation, snow, wind direction, wind speed, temperature, humidity, UV Index, dew point, etc. Data for these environmental factors may be generated via external sources, as discussed in more detail below.

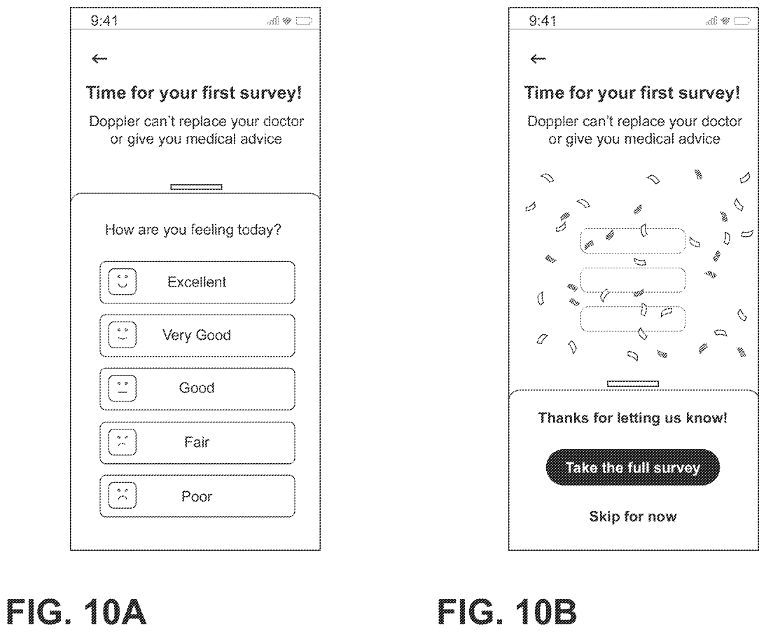

As previously discussed with regard to the exemplary web application above, the mobile application may be configured to collect and aggregate a user's response to surveys regarding the user's health. The application may further be configured to directly provide the surveys to the user. The user may be prompted to select a long survey or a short survey. If the user selects the short survey, the user may be prompted to select an overall health or feeling from a list (e.g., excellent, very good, good, fair, poor). FIGS. 10A and 10B show frames from an exemplary short survey that may be delivered to the user via the mobile application. It will be understood that the surveys may be generic surveys or may be otherwise customized to the user. For example, the surveys may be configurable or customizable to a user, the user's location, the user's health condition, etc.

In various examples, the application may be further configured to provide a user a space to record notes that may be stored within the application or within the application architecture. For example, the application may include a notes section, an input section, or a journal section.

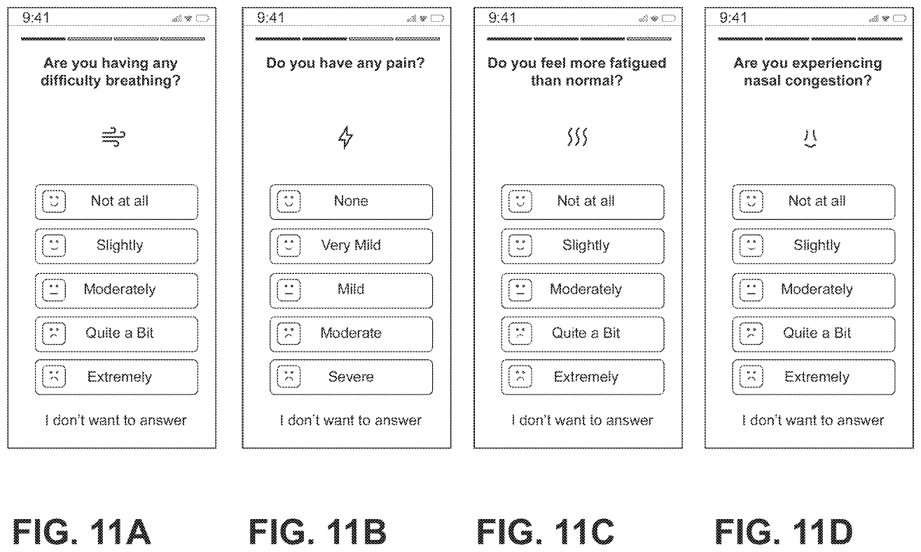

After the user inputs a response, the application may be configured to prompt the user to proceed to a longer survey. The user may select to skip the longer survey or to proceed with providing additional responses to the longer survey. If the user elects to provide responses to the full survey, the user may be prompted to answer questions regarding specific conditions or symptoms. As noted above, the specific condition or symptoms targeted by the long survey may be specific to (e.g., configurable and customizable for) the user. For example, the user may be prompted to indicate breathing difficulty, pain, fatigue, congestion, etc. FIGS. 11A-11D illustrate frames from an exemplary long survey that may be deliver to the user via the mobile application. As discussed in more detail below, the application may be configured to log the responses as user input and may be configured to utilize the system, including various algorithms, to correlate the user data with the collected information (e.g., the environmental factors).

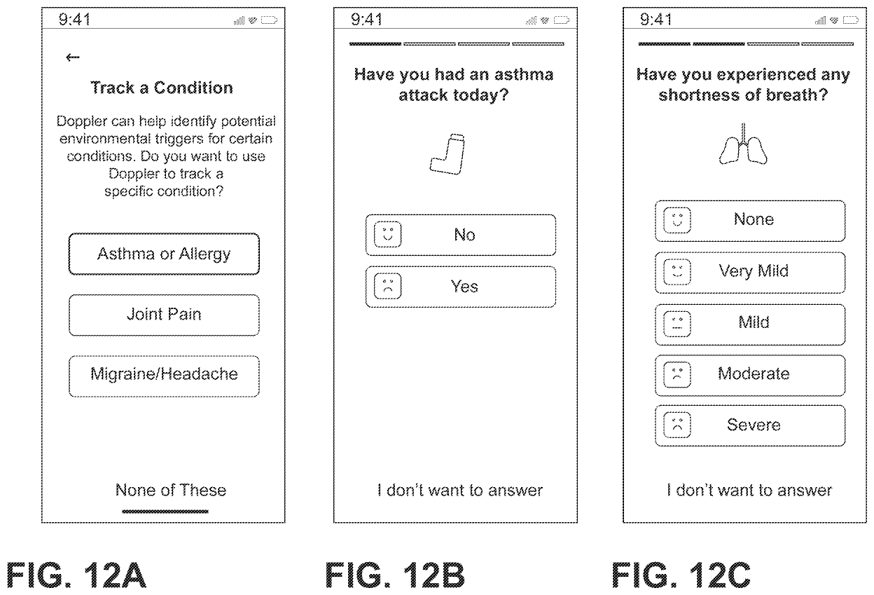

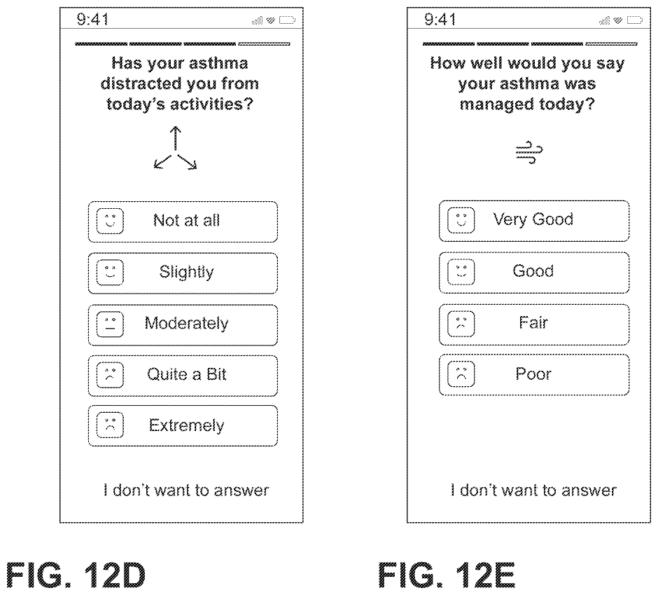

The application may be further configured to provide one or more user surveys tailored for a respective specific health condition (e.g., asthma, joint pain, headaches). These surveys may be configured to allow the user to track symptoms of the specific health condition. In other words, the application may be configured to provide surveys specific to the symptoms related to the specific health condition. For example, if a user has indicated that he or she has asthma, the application may be configured to prompt questions regarding whether the user has had an asthma attack, experienced shortness of breath, had difficulty falling asleep, the user's perceived management level of the asthma, etc. FIG. 12A shows a selection page for selecting a condition specific user survey, and FIGS. 12B-12E show various questions for an exemplary condition specific user survey. In various examples, the application may also be configured to include information regarding each environmental factors and how it may relate to a user's health. The application may be further configured to direct a user to additional information if the user wants to learn more about an environmental factor.

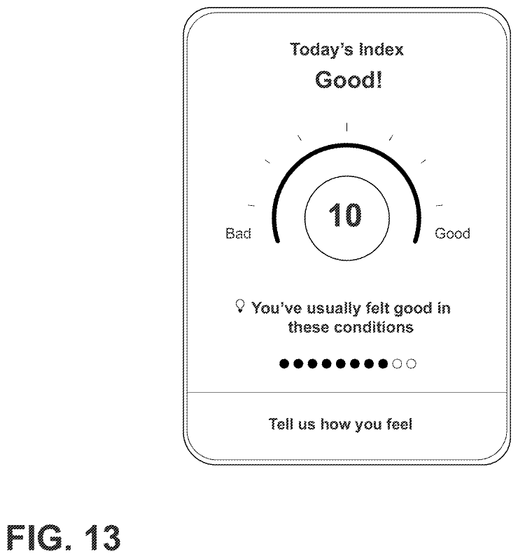

As previously noted, the application may be configured to utilize machine learning within the system to compare the user input with the collected information, including the collected information regarding environmental factors. The application may be configured to display a scaled index score generated from a statistical model utilized by the machine learning, as discussed in more detail elsewhere herein. In various examples, the scaled index score may be a numerical representation. With reference now to FIG. 13, the scaled index score of a single day may be displayed within the application.

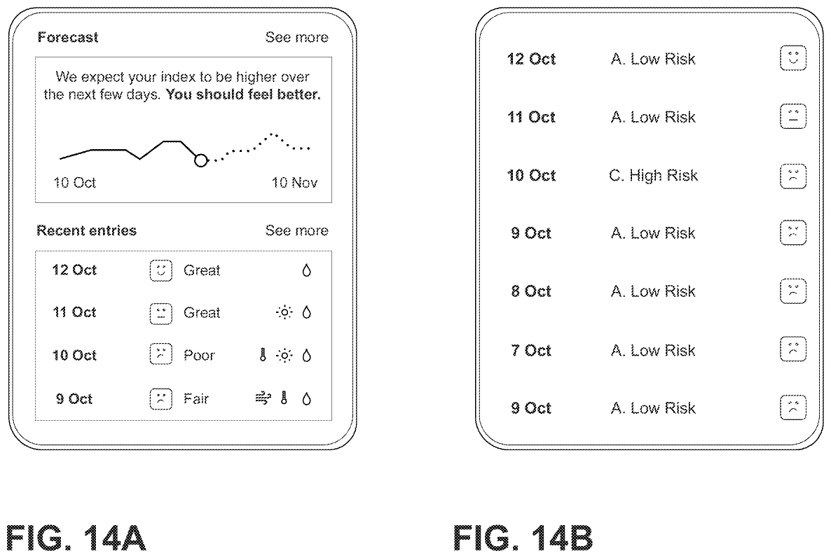

The application may be configured to store and/or aggregate health data for a user. The health data may be generated from the application (e.g., may be obtained from the user) or may be collected from other health related applications on the mobile device. The health data may also be collected from wearable devices configured to pair with the mobile device the application is stored on (e.g., fitness trackers, pedometers, wearable mobile devices, or other wearable devices). Users can use the application to access the health data including, but not limited to, a user's vitals, sleep data, mood data, previous scaled index scores from various days, charts, and/or graphs indicated potential problem days and/or environments based on anticipated environmental factors. For example, the application may be configured to display the daily scaled index score numerically, graphically, or as a combination thereof. As shown in FIGS. 14A and 14B, the scaled index score of multiple days and/or user survey responses may be displayed within the application. The scaled index scores may be displayed as a graph or chart (FIG. 14A). In other examples, the user survey responses may be displayed in a separate list. All or a portion of the user survey responses used to generate the graph or chart may be displayed below the respective graph or chart. (FIG. 14A), or the user survey responses may be displayed in a separate toggleable screen (FIG. 14B). Each of the user survey responses provided in the list may have the date of the response, the user survey response, a visual representation (e.g., a smiling face or a frowning face), a risk level, and/or a visual representation of environmental factors logged in association with the respective user survey response. In various examples, the list may be chronologically ordered. It will be understood that other information logged by the application and associated with the user survey responses and/or the scaled index score may be displayed in the graph, chart, or list without departing from the scope of the present disclosure.

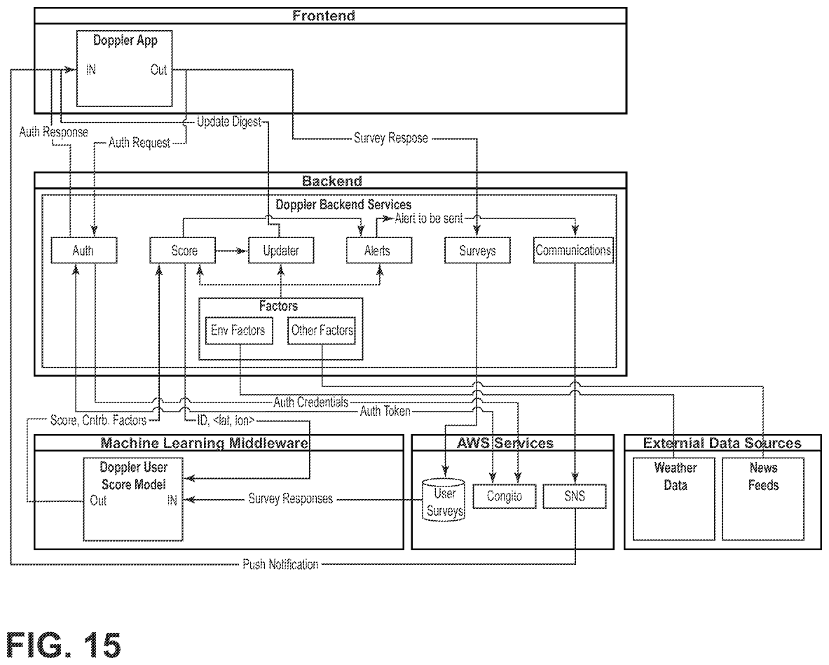

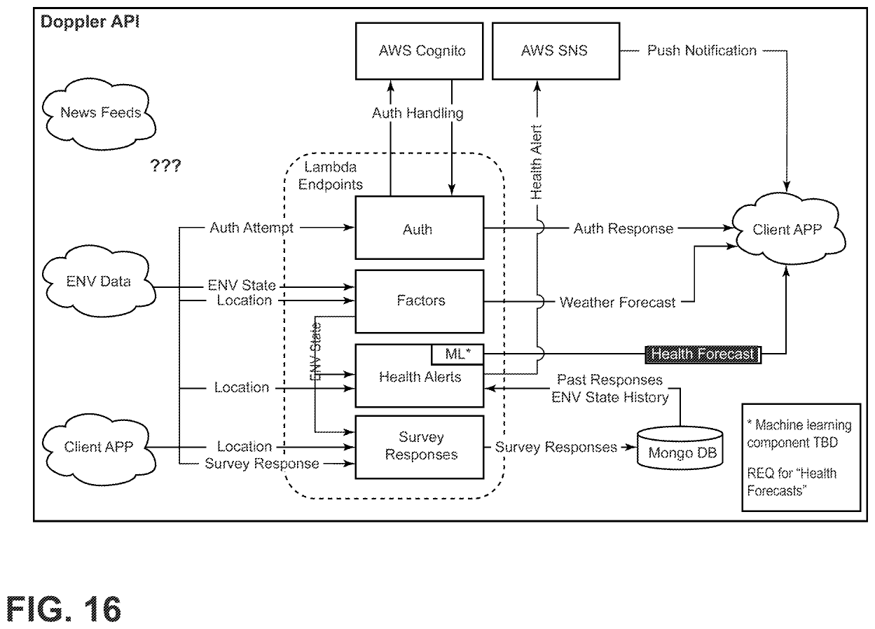

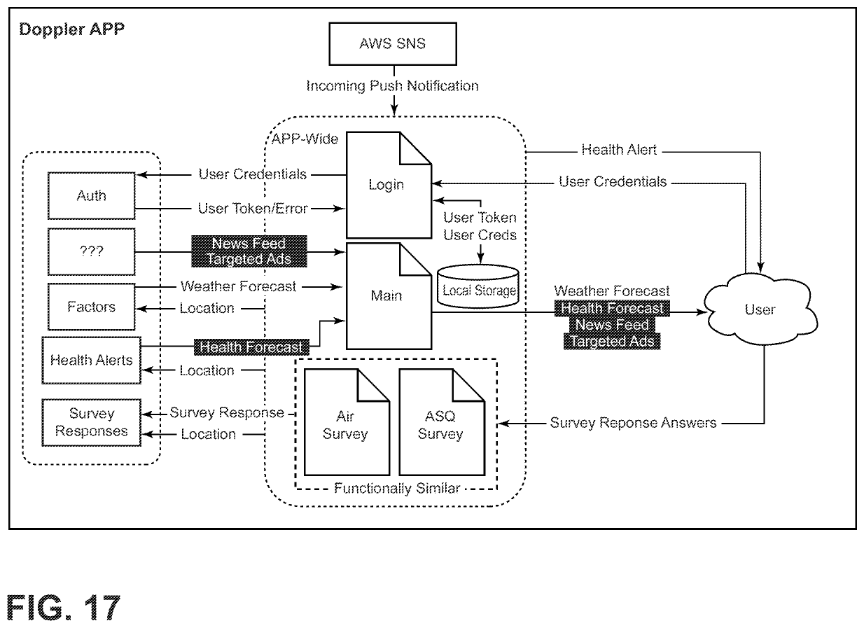

As previously noted, the application may be a component of an overall health monitoring system. As shown in FIGS. 15-17, the overall system may include the application in communication with one or more backend service components, which may be generally formed of one or more modules, external data sources, cloud-based service components, and a machine learning middleware component. The application is configured to be in communication with the backend service components or modules, and the cloud-based service component. FIG. 15 illustrates a block diagram of

Referring still to FIGS. 15-17, the external data sources may be configured to provide data regarding the environmental state at the user's location (e.g., weather data), data from a user's wearable device or other health data stored in other mobile applications, and news to one or more of the backend service components or modules. For example, as previously discussed, the external data sources may be news or weather sources that provide public bulk data for analyzing. The external data sources are configured to provide environmental data to a factor module to be analyzed and included as factors for calculating the scaled index score, as discussed in more detail elsewhere herein.

In various examples, data from the external data sources may be used to log a user's location for future reference. The logged location information may be configured to be stored within the application to generate specific data, news, and health data based on a user's location. For example, if a user intends to travel to a location, the specific data, news, and health data may be tailored to the location to inform a user how the user may feel and what the user should pack (e.g., medications, medical devices, additional clothing, etc.) for the user's time at that location. It is contemplated that any number of locations may be logged. It is also contemplated that wearable devices, as discussed above, may be used to generate logged location information in conjunction with and/or in place of external data sources without departing from the scope of the present disclosure.

One or more of the backend service components or modules may be configured to be in communication with the application, the machine learning middleware component, the cloud-based service component, and/or the external data service components. For example, one or more of the backend service components or modules may be configured to store backend data for utilization of the mobile application including, but not limited to, data such as surveys (e.g., a survey module), data from the external data sources (e.g., the factor module), user information, etc. The backend service components or modules may further include a score module configured to aggregate information from user responses to surveys and scaled index score information, as discussed in more detail elsewhere herein. The score module may also be configured to send and receive information to the middleware component related to the scaled index score.

The aggregated information may be provided to an updater module configured to create updates for delivery to the application and/or may be utilized to generate alerts to be sent to the user via the application. The updates may be delivered to the application via a news feed, digest, or other updatable information stream. In various examples, the news feed, digest, or other updatable information stream may also be configured to display targeted ads based on the user survey responses, environmental data, and any other data provided to or logged by the application.

One or more of the backend service components or modules may further be in communication with a cloud-based service component (e.g., Amazon Web Services “AWS”). The cloud-based service component may include a storage service for user responses to surveys. When a user provides responses to a survey, the user responses are provided to a survey module which communicates the data to the storage service. The data may be later used for calculating information for delivery to the user, as discussed in more detail elsewhere herein. While the backend service components are described herein a modules, it will be understood than any other backend service component configured to provide or perform the same or similar function may be utilized without departing from the scope of the present disclosure.

With continued reference to FIGS. 15-17, the cloud-based service component may further include a user identity and data synchronization service (e.g., Amazon Cognito). For example, the backend service components or modules may include an authorization module in communication with the application and the user identity and data synchronization service. The application is configured to provide an authorization request to the authorization module. The authorization module is configured to communicate authorization credentials to the user identity and data synchronization service. The user identity and data synchronization service may be configured to respond with an authorization token which is received by the authorization module. The authorization module may then be configured to provide an authorization response to the application.

The cloud-based service component may further include a simple notification service (“SNS”) configured to provide application to application and/or application to person notifications (e.g., Amazon Simple Notification Service). For example, the SNS to enable dissemination of messages to one or more users via SMS messaging, push notifications, and/or email. The SNS may be configured to communicated with a communications module of the backend service components. The communications module may be configured to receive data from the alerts module to provide to the SNS to deliver notifications, such as, for example, push notifications via the application.

User responses and environmental data from the external sources may be aggregated to create a statistical model for anticipated symptoms or changes in conditions and overall feeling based on environmental changes. The software system may further include the statistical model for use in calculating the scaled index score. The middleware component is configured to receive score information, including information regarding environmental factor and other informational data, from the backend service components or modules and is configured to receive survey responses from the cloud-based service component. These inputs are analyzed by the model and communicated to the backend service components or modules for delivery to the user via the application for user review. For example, as previous discussed, the application may be configured to generate the scaled index score to indicate anticipated problems and overall feeling based on present environmental conditions.

The scaled index score is a probability of some environmental event triggering acute symptoms in the user. The higher the scaled index score is, the more likely it is that the day will not result in the user exhibiting acute symptoms. The scaled index score may be generated utilizing a model that attributes a health condition y as being a function of a user's genetics g, the user's behavior b, the user's conditions c, and the user's environment x. The model may be represented by a function:

y=f(g⋄b⋄c⋄x)

The behavior variable may include health-affecting behaviors such as smoking, working out, drinking soft drinks, etc. The conditions variable is configured to account for conditions such as age, socioeconomic demographics, and/or already developed health conditions (e.g., immunocompromised). Specific formulae may be used in conjunction with machine learning to refine the scaled index score.

We anticipate that this disclosed invention has several potential uses. For example, it could be useful to Public Health Organizations as an outbreak investigation and containment tool, to health insurers as an actuarial investigation tool, and/or to financial institutions to gain insights for site selection and financial modeling. Schools and companies could be interested in the tool for tracking and increasing attendance. It can also be used as a diagnostic tool. The application may further be used as a self-monitoring tool for users to determine and predict impacts of the environment and/or their location on their health, overall feeling, and well-being.

According to at least one aspect, a software system may include an application configured to receive user input, a cloud-based service component in communication with the application, a middleware component in communication with the cloud-based service component, and a plurality of backend modules in communication with at least one of the application, the middleware component, and the cloud-based service component. A machine learning component may be stored on the middleware component. The machine learning component may be configured to analyze the user input and environmental data to determine a probability of a user health condition. It will be understood that the software system may include both software components and hardware components.

According to another aspect, a plurality of backend modules may include a factor module, an updater module, and a score module. The factor module may be configured to communicate received data to the updater module and the score module.

According to another aspect, an external data source may be configured to provide external data to a factor module. The external data may be a portion of received data.

According to another aspect, an external data source may be a wearable device configured to monitor, record, or otherwise utilize and/or store user health data.

According to another aspect, external data may be one of environmental data, user-specific health data, and news data.

According to another aspect, a machine learning component may be one of a machine learning algorithm, a machine learning layer, and a machine learning subsystem.

According to another aspect, an application may be configured to provide a user one or more surveys. In various examples, the one or more surveys may be customizable. In various examples, the one or more surveys may be configurable.

According to another aspect, an application may be one of a web application and a mobile application.

According to another aspect, a method for analyzing health data may include providing a user one or more surveys. The one or more surveys may be customizable and/or configurable. The method may include compiling user input from the one or more surveys and/or may include collecting information regarding external factors. The method may include analyzing the user input relative to the information regarding the external factors and/or may include generating output for the user based on the analysis of the user input relative to the information regarding the external factors.

According to another aspect, one of a mobile application and a web application may be utilized to provide a user one or more surveys.

According to another aspect, a method for analyzing health data may include displaying an output for use by a user.

According to another aspect, a method for analyzing health data may include calculating a probability of user symptoms using output from an analysis.

According to another aspect, a method for analyzing health data may include calculating a probability of user symptoms using the output from the analysis and/or utilizing a machine learning algorithm to generate a scaled numerical score.

According to another aspect, a method for analyzing health data may include assigning a scaled numerical score to a probability or user symptoms.

According to another aspect, a method for analyzing health data may include utilizing a cloud-based service component to generate notifications based on output for a user.

According to another aspect, a method for analyzing health data may include utilizing an application to provide a user one or more surveys, and/or compiling user input from the one or more surveys. The method may include collecting information regarding environmental factors and/or analyzing the user input relative to the information regarding the environmental factors. The method may include generating output for the user based on the analysis of the user input relative to the information regarding the environmental factors and/or displaying the output for the user within the application.

According to another aspect, a method for analyzing health data may include collecting location data and/or location-specific environmental data. The location data and/or the location-specific environmental data may be used to predict location specific output.

According to another aspect, one of a mobile application and a web application may be utilized to provide a user one or more surveys. The one or more surveys may be customizable and/or configurable.

According to another aspect, a method for analyzing health data may displaying output as a graph.

According to another aspect, a method for analyzing health data may include displaying health data as a chronological list of user inputs and/or associated environmental factors.

According to another aspect, a method for analyzing health data may include analyzing user input relative to information regarding environmental factors and/or utilizing a machine learning algorithm to generate a scaled numerical score.

According to another aspect, a method for analyzing health data may include generating a scaled numerical score via a machine learning algorithm based on a statistical model determining probability.

According to another aspect, a method for analyzing health data may include generating a scaled numerical score via a machine learning algorithm based on a statistical model prediction.

The terms “comprising,” “including,” and “having,” as used in the claims and specification herein, shall be considered as indicating an open group that may include other elements not specified. The terms “a,” “an,” and the singular forms of words shall be taken to include the plural form of the same words, such that the terms mean that one or more of something is provided. The term “one” or “single” may be used to indicate that one and only one of something is intended. Similarly, other specific integer values, such as “two,” may be used when a specific number of things is intended. The terms “preferably,” “preferred,” “prefer,” “optionally,” “may,” and similar terms are used to indicate that an item, condition or step being referred to is an optional (not required) feature of the invention.

The invention has been described with reference to various specific and preferred embodiments and techniques. However, it should be understood that many variations and modifications may be made while remaining within the spirit and scope of the invention. It will be apparent to one of ordinary skill in the art that methods, devices, device elements, materials, procedures and techniques other than those specifically described herein can be applied to the practice of the invention as broadly disclosed herein without resort to undue experimentation. All art-known functional equivalents of methods, devices, device elements, materials, procedures and techniques described herein are intended to be encompassed by this invention. Whenever a range is disclosed, all subranges and individual values are intended to be encompassed. This invention is not to be limited by the embodiments disclosed, including any shown in the drawings or exemplified in the specification, which are given by way of example and not of limitation.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

All references throughout this application, for example patent documents including issued or granted patents or equivalents, patent application publications, and non-patent literature documents or other source material, are hereby incorporated by reference herein in their entireties, as though individually incorporated by reference, to the extent each reference is at least partially not inconsistent with the disclosure in the present application (for example, a reference that is partially inconsistent is incorporated by reference except for the partially inconsistent portion of the reference).

What is claimed:

1. A software system comprising:
an application configured to receive user input;
a cloud-based service component in communication with the application;
a middleware component in communication with the cloud-based service component;
a machine learning component stored on the middleware component, the machine learning component configured to analyze the user input and external data to determine a probability of a user health condition; and
a plurality of backend modules in communication with at least one of the application, the middleware component, and the cloud-based service component.

2. The software system of claim 1, wherein the plurality of backend modules includes a factor module, an updater module, and a score module, and further wherein the factor module is configured to communicate received data to the updater module and the score module.

3. The software system of claim 2 further comprising:
an external data source configured to provide the external data to the factor module, wherein the external data is a portion of the received data.

4. The software system of claim 3, wherein the external data is one of environmental data, user-specific health data, and news data.

5. The software system of claim 3, wherein the machine learning component is one of a machine learning algorithm, a machine learning layer, and a machine learning subsystem.

6. The software system of claim 1, wherein the application is configured to provide a user one or more surveys.

7. The software system of claim 1, wherein the application is one of a web application and a mobile application.

8. A method for analyzing health data comprising steps of:
providing a user one or more surveys;
compiling user input from the one or more surveys;
collecting information regarding external factors;
analyzing the user input relative to the information regarding the external factors; and
generating output for the user based on the analysis of the user input relative to the information regarding the external factors.

9. The method of claim 8, wherein one of a mobile application and a web application is utilized to provide the user the one or more surveys.

10. The method of claim 8, further comprising a step of:
displaying the output for use by the user.

11. The method of claim 8, further comprising a step of:
calculating a probability of user symptoms using the output from the analysis.

12. The method of claim 11, wherein calculating a probability of user symptoms using the output from the analysis further includes utilizing a machine learning algorithm to generate a scaled numerical score.

13. The method of claim 12, further comprising a step of:
assigning the scaled numerical score to the probability.

14. The method of claim 8, further comprising a step of:
utilizing a cloud-based service component to generate notifications based on the output for the user.

15. A method for analyzing health data comprising steps of:
utilizing an application to provide a user one or more surveys;
compiling user input from the one or more surveys;
collecting information regarding environmental factors;
analyzing the user input relative to the information regarding the environmental factors;
generating output for the user based on the analysis of the user input relative to the information regarding the environmental factors; and
displaying the output for the user within the application.

16. The method of claim 15, wherein one of a mobile application and a web application is utilized to provide the user one or more surveys.

17. The method of claim 15, wherein the environmental factors are location specific environmental factors and output is generated for a location based on the location specific environmental factors.

18. The method of claim 15, wherein the output is displayed as one of a graph and a chronological list of user inputs and associated environmental factors.

19. The method of claim 15, wherein analyzing the user input relative to the information regarding the environmental factors further includes utilizing a machine learning algorithm to generate a scaled numerical score.

20. The method of claim 19, wherein the scaled numerical score is generated by the machine learning algorithm based on a statistical model prediction.
